(19) United States
(12) Patent Application Publication
(10) Pub. No.: US 2010/0261154 A1
(43) Pub. Date: Oct. 14, 2010

(54) PRIMERS AND PROBES FOR DETECTING HEPATITIS C VIRUS

(75) Inventors: Claudia Esping, Geneva, IL (US); George Schneider, Barrington, IL (US)

Correspondence Address: VYSIS, INC;PATENT DEPARTMENT, 1300 E TOUHY AVENUE, DES PLAINES, IL, 60018, US

(73) Assignee: ABBOTT LABORATORIES, Abbott Park, IL (US)

(21) Appl. No.: 12/648,311

(22) Filed: Dec. 29, 2009

Related U.S. Application Data
(60) Provisional application No. 61/141,850, filed on Dec. 31, 2008.

(51) Int. Cl. C12Q 1/70; C07H 21/04

(52) U.S. Cl. 435/5; 536/24.33; 536/24.32

(57) ABSTRACT

The present invention relates to primers, probes, primer sets, primer and probe sets, methods and kits for detecting Hepatitis C virus in a test sample.

### RELATED APPLICATION INFORMATION

This application claims priority from U.S. Ser. No. 61/141,850, filed on Dec. 31, 2008, the contents of which are herein incorporated by reference.

### TECHNICAL FIELD

The present invention relates to primers, probes, primer sets, primer and probe sets, methods and kits for detecting Hepatitis C virus in a test sample.

### BACKGROUND

Hepatitis C virus (HCV) is a member of the Hepacivirus genus of the Flaviviridae family. There are currently 6 recognized clades of HCV that differ from each other by approximately 25-35% at the nucleotide level. Representative genotypes of HCV include HCV-10, HCV-11, HCV-1a, HCV-1b, HCV-2a, HCV-2b, HCV-3a, HCV-4-a, HCV-5a, and HCV-6a. HCV is recognized as the principal agent of parenterally transmitted non-A, non-B hepatitis. Chronic infection with HCV may lead to chronic hepatitis, cirrhosis, and hepatocellular carcinoma. HCV infection usually occurs through contact with infected blood, for example, through intravenous drug use, but HCV can be sexually transmitted as well as passed from mother to child during childbirth.

Around 3% (170 million) of the world's population has been infected with HCV. Though acute HCV infections are asymptomatic or cause mild clinical illness, chronic HCV infection develops in 75%-85% of those acutely infected, with chronic liver disease developing in 60%-70% of chronically infected persons (CDC. Recommendations for prevention and control of hepatitis C Virus (HCV) infection and HCV-related chronic disease. Morbid Mortal Wkly Rep 1998, 47(RR-19):1-39). Chronic hepatitis C is the leading cause for liver transplantation in the United States. Methods for accurate detection of HCV would provide a powerful tool to aid in the prevention and treatment of HCV infections.

The HCV genome includes a 9.6-kb molecule of linear positive-sense, single-stranded RNA, which encodes a large polyprotein of about 3010-3033 amino acids (Choo et al., Science (1989) 244, 359-362; Kato et al., Proc. Natl. Acad. Sci. USA (1990) 87, 9524-9528). The HCV genome exhibits considerable sequence diversity among genotypes, however, the 5′-untranslated region (UTR) and the 3′-UTR are relatively highly conserved, suggesting that these regions have may an important functional role in the regulation of replication, translation, and/or packing processes (Ito et al., Virology (1999) 254, 288-296; Yamada et al., Virology (1996) 223, 255-261). The 5′-UTR includes an internal ribosome entry site and binding sites for numerous host cell factors that may regulate HCV genome translation (Ito et al., Virology (1999) 254, 288-296). The 3′-UTR region includes three domains: a highly variable sequence of 21 to 39 nucleotides (nt); followed by a UC-rich sequence of variable length (73 to 98 nt); and a distal 3′ 98 nt highly conserved sequence (Ito et al., J. Virol. (1997) 71 (11), 8698-8706).

A variety of oligonucleotide-based methods for detecting HCV have been devised. U.S. Pat. No. 5,714,596 teaches the use of oligomers specific for coding sequences conserved among HCV and flaviviruses for polymerase chain reaction (PCR)-based and probe hybridization assays for identifying HCV variants in a sample. Once it was recognized that the 5′-UTR and 3′-UTR regions were conserved, these regions became targets of interest for assays for detecting HCV in samples. For example, U.S. Pat. No. 5,837,442 teaches oligonucleotide primers for reverse transcriptase PCR (RT-PCR) amplification of a region of the 5′-UTR of HCV. As well, U.S. Pat. No. 6,297,003 teaches oligonucleotide primers and probes targeting the 3′-UTR to screen for complementary sequences and related clones in the same or alternate species. Furthermore, a number of manufacturers have developed various HCV assays including: the RealTime HCV assay (Abbott Laboratories, DesPlaines, Ill.), which targets the 5′-UTR of HCV to detect HCV in human serum and plasma from HCV-infected individuals; the Bayer Versant™ HCV RNA 3.0 Assay (Bayer Diagnostics, Berkeley, Calif.), which uses branched DNA labeled probes to detect HCV by targeting the 5′-UTR and core regions; the Cobas Amplicor HCV Monitor version 2.0 assay (Roche Diagnostic Systems, Branchburg, N.J.) and the Chiron Quantiplex (Chiron, Emeryville, Calif.) assays, which target the 5′-UTR; and the Apath 3′-UTR (Apath, LLC, St. Louis, Mo.) and EraGen HCV (EraGen Biosciences, Madison, Wis.) assays, which use quantitative RT-PCR to target the 3′-UTR of HCV. However, despite such methods, there remains a need for new methods that provide greater clinical sensitivity and specificity in a high throughput and efficient workflow environment.

### SUMMARY

In one embodiment, the present invention relates to a primer for amplifying Hepatitis C virus (HCV) in a test sample. The primer has a sequence selected from the group consisting of: SEQ ID NO:1, SEQ ID NO:2, SEQ ID NO:3, SEQ ID NO:4, SEQ ID NO:5, and SEQ ID NO:6, and complements thereof.

In another embodiment, the present invention relates to a probe for detecting HCV in a test sample. The probe has a sequence selected from the group consisting of: SEQ ID NO:7, SEQ ID NO:8, and complements thereof.

In still yet a further embodiment, the present invention relates to a primer set for amplifying HCV in a test sample. The primer set includes:
(a) at least one forward primer having a sequence selected from the group consisting of: SEQ ID NO:1, SEQ ID NO:2, complements thereof, and any combinations thereof; and
(b) at least one reverse primer having a sequence selected from the group consisting of: SEQ ID NO:3, SEQ ID NO:4, SEQ ID NO:5, SEQ ID NO:6, complements thereof, and any combinations thereof.

In still yet a further embodiment, the present invention relates to a method for detecting HCV in a test sample. The method comprising the steps of:
(a) contacting a test sample with at least one forward primer having a sequence selected from the group consisting of SEQ ID NO:1 and SEQ ID NO:2, or complements thereof and at least one reverse primer having a sequence selected from the group consisting of SEQ ID NO:3, SEQ ID NO:4, SEQ ID NO:5, and SEQ ID NO:6 or complements thereof under amplification conditions to generate a first target sequence; and
(b) detecting hybridization between the first target sequence and at least one probe as an indication of the presence of HCV in the test sample, wherein the at least one probe has a sequence selected from the group consisting of: SEQ ID NO:7 and SEQ ID NO:8, or complements thereof.

In the above described method, the amplification conditions comprise submitting the test sample to an amplification reaction carried out in the presence of suitable amplification reagents. Additionally, the amplification reaction can comprise using at least one of PCR, real-time PCR (such as, but not limited to, a Taq-Man® assay), or reverse-transcriptase PCR (RT-PCR).

In the above described method, at least one probe is labeled with a detectable label. As is known in the art, the detectable label can be directly attached to at least one probe. Moreover, the detectable label can be directly detectable. For example, the detectable label can comprise a fluorescent moiety attached at the 5′ end of at least one probe. Moreover, at least one probe can further comprise a quencher moiety attached at its 3′ end. The detectable label and quencher moiety may be interchanged between the 5′ and the 3′ ends. It is further contemplated herein that while the probe is not bound to its target sequence, the detectable label and quencher moiety are reversibly maintained within such proximity that the quencher blocks the detection of the detectable label. Quantification of detected label enables determination of target HCV copy numbers. Quantification assays contemplated for use herein include, for example, the TaqMan® assay, hybridization protection assays, and heterogeneous detection systems, to name a few.

Alternatively, the detectable label can be indirectly attached to at least one probe. Alternatively, the detectable label can be indirectly detectable.

In addition, the above described method can comprise the steps of:

(a) contacting the test sample with a forward primer having a sequence of SEQ ID NO:1 or a complement thereof and a reverse primer having a sequence of SEQ ID NO:3 or complement thereof under amplification conditions to generate a second target sequence; and

(b) detecting hybridization between the second target sequence and a probe having a sequence of SEQ ID NO:7 or a complement thereof as an indication of the presence of HCV in the test sample.

In still another aspect, the present invention relates to a kit for detecting HCV in a test sample. The kit comprises:

(a) at least one forward primer having a sequence selected from the group consisting of: SEQ ID NO:1, SEQ ID NO:2, complements thereof, and any combinations thereof;

(b) at least one reverse primer having a sequence selected from the group consisting of: SEQ ID NO:3, SEQ ID NO:4, SEQ ID NO:5, SEQ ID NO:6, complements thereof, and any combinations thereof; and

(c) amplification reagents.

The above described kit can also further comprise at least one probe, wherein the at least one probe is selected from the group consisting of: SEQ ID NO:7, SEQ ID NO:8, and complements thereof.

### DETAILED DESCRIPTION

The present invention relates to primers, probes, primer sets and primer and probe sets that can be used to amplify and/or detect HCV in a test sample. The present invention also relates to methods of detecting HCV in test samples using the primer and probe sets described herein. The present invention also relates to kits for detecting HCV sequences in a test sample.

The primer and probe sets of the present invention achieve robust clinical sensitivity and specificity. Finally, the primer and probe sets of the present invention provide high throughput and efficient workflow.

### A. DEFINITIONS

As used herein, the singular forms “a,” “an,” and “the” include plural referents unless the context clearly dictates otherwise. For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

a) Amplicon

As used herein, the term “amplicon” refers to a product of an amplification reaction. An example of an amplicon is a DNA or an RNA product (usually a segment of a gene, DNA or RNA) produced as a result of PCR, real-time PCR, RT-PCR, competitive RT-PCR, ligase chain reaction (LCR), gap LCR, strand displacement amplification (SDA), nucleic acid sequence based amplification (NASBA), transcription-mediated amplification (TMA), or the like.

b) Amplification, Amplification Method, or Amplification Reaction

As used herein, the phrases “amplification,” “amplification method,” or “amplification reaction,” are used interchangeably and refer to a method or process that increases the representation of a population of specific nucleic acid (all types of DNA or RNA) sequences (such as a target sequence or a target nucleic acid) in a test sample. Examples of amplification methods that can be used in the present invention include, but are not limited to, PCR, real-time PCR, RT-PCR, competitive RT-PCR, and the like, all of which are known to one skilled in the art.

c) Amplification Conditions

As used herein, the phrase “amplification conditions” refers to conditions that promote annealing and/or extension of primer sequences. Such conditions are well-known in the art and depend on the amplification method selected. For example, PCR amplification conditions generally comprise thermal cycling, e.g., cycling of the reaction mixture between two or more temperatures. In isothermal amplification reactions, amplification occurs without thermal cycling although an initial temperature increase may be required to initiate the reaction. Amplification conditions encompass all reaction conditions including, but not limited to, temperature and temperature cycling, buffer, salt, ionic strength, pH, and the like.

d) Amplification Reagents

As used herein, the phrase “amplification reagents” refers to reagents used in amplification reactions and may include, but is not limited to, buffers, reagents, enzymes having reverse transcriptase, and/or polymerase, or exonuclease activities; enzyme cofactors such as magnesium or manganese; salts; and deoxynucleotide triphosphates (dNTPs), such as deoxyadenosine triphosphate (dATP), deoxyguanosine triphosphate (dGTP), deoxycytidine triphosphate (dCTP), deoxythymidine triphosphate (dTTP), and deoxyuridine triphosphate (dUTP). Amplification reagents may readily be selected by one skilled in the art depending on the amplification method employed.

e) Directly Detectable and Indirectly Detectable

As used herein, the phrase, “directly detectable,” when used in reference to a detectable label or detectable moiety, means that the detectable label or detectable moiety does not require further reaction or manipulation to be detectable. For example, a fluorescent moiety is directly detectable by fluorescence spectroscopy methods. In contrast, the phrase “indirectly detectable,” when used herein in reference to a detectable label or detectable moiety, means that the detectable label or detectable moiety becomes detectable after further reaction or manipulation. For example, a hapten becomes detectable after reaction with an appropriate antibody attached to a reporter, such as a fluorescent dye.

f) Fluorophore, Fluorescent Moiety, Fluorescent Label, or Fluorescent Dye

The terms, “fluorophore,” “fluorescent moiety,” “fluorescent label,” and “fluorescent dye” are used interchangeably herein and refer to a molecule that absorbs a quantum of electromagnetic radiation at one wavelength, and emits one or more photons at a different, typically longer, wavelength in response thereto. Numerous fluorescent dyes of a wide variety of structures and characteristics are suitable for use in the practice of the present invention. Methods and materials are known for fluorescently labeling nucleic acid molecules (See, R. P. Haugland, “Molecular Probes: Handbook of Fluorescent Probes and Research Chemicals 1992-1994,” 5th Ed., 1994, Molecular Probes, Inc.). Preferably, a fluorescent label or moiety absorbs and emits light with high efficiency (e.g., has a high molar absorption coefficient at the excitation wavelength used, and a high fluorescence quantum yield), and is photostable (e.g., does not undergo significant degradation upon light excitation within the time necessary to perform the analysis). Rather than being directly detectable themselves, some fluorescent dyes transfer energy to another fluorescent dye in a process called fluorescence resonance energy transfer (FRET), and the second dye produces the detected signal. Such FRET fluorescent dye pairs are also encompassed by the term “fluorescent moiety.” The use of physically-linked fluorescent reporters/quencher moieties is also within the scope of the present invention. In these aspects, when the fluorescent reporter and quencher moiety are held in close proximity, such as at the ends of a probe, the quencher moiety prevents detection of a fluorescent signal from the reporter moiety. When the two moieties are physically separated, such as after cleavage by a DNA polymerase, the fluorescent signal from the reporter moiety becomes detectable.

g) Hybridization

As used herein, the term “hybridization” refers to the formation of complexes between nucleic acid sequences which are sufficiently complementary to form complexes via Watson-Crick base pairing or non-canonical base pairing. For example, when a primer “hybridizes” with a target sequence (template), such complexes (or hybrids) are sufficiently stable to serve the priming function required by, e.g., the DNA polymerase, to initiate DNA synthesis. It will be appreciated by one skilled in the art that hybridizing sequences need not have perfect complementarity to provide stable hybrids. In many situations, stable hybrids will form where fewer than about 10% of the bases are mismatches. Accordingly, as used herein, the term “complementary” refers to an oligonucleotide that forms a stable duplex with its complement under assay conditions, generally where there is about 80%, about 81%, about 82%, about 83%, about 84%, about 85%, about 86%, about 87%, about 88%, about 89%, about 90%, about 91%, about 92%, about 93%, about 94% about 95%, about 96%, about 97%, about 98%, or about 99% greater homology. Those skilled in the art understand how to estimate and adjust the stringency of hybridization conditions such that sequences having at least a desired level of complementarity will stably hybridize, while those having lower complementarity will not. Examples of hybridization conditions and parameters can be found, for example in, Sambrook et al., “Molecular Cloning: A Laboratory Manual,” 1989, Second Edition, Cold Spring Harbor Press: Plainview, N.Y.; F. M. Ausubel, “Current Protocols in Molecular Biology,” 1994, John Wiley & Sons: Secaucus, N.J.

h) Labeled or Labeled with a Detectable Label

As used herein, the terms “labeled” and “labeled with a detectable label (or agent or moiety)” are used interchangeably herein and specify that an entity (e.g., a primer or a probe) can be visualized, for example following binding to another entity (e.g., an amplification product or amplicon). Preferably, the detectable label is selected such that it generates a signal which can be measured and whose intensity is related to (e.g., proportional to) the amount of bound entity. A wide variety of systems for labeling and/or detecting nucleic acid molecules, such as primer and probes, are well-known in the art. Labeled nucleic acids can be prepared by incorporation of, or conjugation to, a label that is directly or indirectly detectable by spectroscopic, photochemical, biochemical, immunochemical, electrical, optical, chemical, or other means. Suitable detectable agents include, but are not limited to, radionuclides, fluorophores, chemiluminescent agents, microparticles, enzymes, colorimetric labels, magnetic labels, haptens, Molecular Beacons, aptamer beacons, and the like.

i) Primer

The term “primer” refers to an oligonucleotide capable of acting as a point of initiation of synthesis of a primer extension product that is a complementary strand of nucleic acid (all types of DNA or RNA), when placed under suitable amplification conditions (e.g., buffer, salt, temperature and pH) in the presence of nucleotides and an agent for nucleic acid polymerization (e.g., a DNA-dependent or RNA-dependent polymerase). The primer can be single-stranded or double-stranded. If double-stranded, the primer may first be treated (e.g., denatured) to allow separation of its strands before being used to prepare extension products. Such a denaturation step is typically performed using heat, but may alternatively be carried out using alkali, followed by neutralization. The primers of the present invention may have a length of about 15 to about 50 nucleotides in length, preferably from about 20 to about 40 nucleotides in length, most preferably, from about 22 to about 30 nucleotides in length. The primers of the present invention can contain additional nucleotides in addition to those described in more detail herein. For example, primers used in SDA can include a restriction endonuclease recognition site 5′ to the target binding sequence (See, U.S. Pat. Nos. 5,270,184 and 5,455,166), NASBA, and TMA primers can include an RNA polymerase promoter linked to the target binding sequence of the primer. Methods for linking such specialized sequences to a target binding sequence for use in a selected amplification reaction are well known to those skilled in the art.

The phrase “forward primer” refers to a primer that hybridizes (or anneals) with the target sequence (e.g., template strand). The phrase “reverse primer” refers to a primer that hybridizes (or anneals) to the complementary strand of the target sequence. The forward primer hybridizes with the target sequence 5′ with respect to the reverse primer.

j) Primer Set

As used herein, the phrase “primer set” refers to two or more primers which together are capable of priming the amplification of a target sequence or target nucleic acid of interest (e.g., a target sequence within the HCV). In certain embodiments, the term “primer set” refers to a pair of primers including a 5′ (upstream) primer (or forward primer) that hybridizes with the 5′-end of the target sequence or target nucleic acid to be amplified and a 3′ (downstream) primer (or reverse primer) that hybridizes with the complement of the target sequence or target nucleic acid to be amplified. Such primer sets or primer pairs are particularly useful in PCR amplification reactions.

k) Probe

As used herein, the term “probe” refers to an oligonucleotide capable of selectively hybridizing to at least a portion of a target sequence under appropriate hybridization conditions (e.g., a portion of a target sequence that has been amplified). The probes of the present invention have a length of about 10-50 nucleotides, preferably about 12-35 nucleotides and most preferably from 14-25 nucleotides. In certain instances, a probe can be labeled with a detectable label.

l) Primer and Probe Set

As used herein, the phrase “primer and probe set” refers to a combination including two or more primers which together are capable of priming the amplification of a target sequence or target nucleic acid, and least one probe which can detect the target sequence or target nucleic acid. The probe generally hybridizes to a strand of an amplification product (or amplicon) to form an amplification product/probe hybrid, which can be detected using routine techniques known to those skilled in the art.

m) Target Sequence or Target Nucleic Acid

The phrases “target sequence” and “target nucleic acid” are used interchangeably herein and refer to that which the presence or absence of which is desired to be detected. In the context of the present invention, a target sequence preferably includes a nucleic acid sequence to which one or more primers will complex. The target sequence can also include a probe-hybridizing region with which a probe will form a stable hybrid under appropriate amplification conditions. As will be recognized by one of ordinary skill in the art, a target sequence may be single-stranded or double-stranded. In the context of the present invention, target sequences of interest are located within the 3′-UTR of HCV.

n) Test Sample

As used herein, the term “test sample” generally refers to a biological material being tested for and/or suspected of containing an analyte of interest, such as an HCV sequence. The test sample may be derived from any biological source, such as, a cervical, vaginal or anal swab or brush, or a physiological fluid including, but not limited to, whole blood, serum, plasma, interstitial fluid, saliva, ocular lens fluid, cerebral spinal fluid, sweat, urine, milk, ascites fluid, mucus, nasal fluid, sputum, synovial fluid, peritoneal fluid, vaginal fluid, menses, amniotic fluid, semen, and so forth. The test sample may be used directly as obtained from the biological source or following a pretreatment to modify the character of the sample. For example, such pretreatment may include preparing plasma from blood, diluting viscous fluids, and so forth. Methods of pretreatment may also involve filtration, precipitation, dilution, distillation, mixing, concentration, lyophilization, inactivation of interfering components, the addition of reagents, lysing, etc. Moreover, it may also be beneficial to modify a solid test sample to form a liquid medium or to release the analyte. Preferably, the sample may be plasma.

### B. PRIMERS, PROBES AND PRIMER AND PROBE SETS

In one embodiment, the present invention relates to one or more primers for amplifying HCV in a test sample. The one or more primers can include a primer having a sequence comprising or consisting of any of the sequences shown below in Table 1, a complement of any of the sequences shown below in Table 1 and any combinations of the sequences shown below in Table 1 and/or their complements. The candidate primer sequences in Table 1 below exhibit cross-genotype specificity, as is shown below in Example 3, Table 9.

TABLE 1

Candidate Primer Sequences.

| | | |
|---|---|---|
| SEQ ID NO: | SEQUENCE (5′ to 3′) | Type of Primer |

| | | |
|---|---|---|
| 1 | gc tcc atc tta gcc cta gtc | Forward Primer |
| 2 | ggc tcc atc tta gcc cta gtc acg | Forward Primer |
| 3 | agc act ctc tgc agt cat gcg gct ca | Reverse Primer |
| 4 | agc act ctc tgc agt cta gcg gct ca | Reverse Primer |
| 5 | agc act ctc tgc agt ctt gcg gct ca | Reverse Primer |
| 6 | agc act ctc tgc agt caa gcg gct ca | Reverse Primer |

In one aspect, the present invention relates to a primer set for amplifying HCV in a test sample containing one or more of the primers described in Table 1. Specifically, the primer set can comprise the following:
(a) at least one forward primer having a sequence selected from the group consisting of: SEQ ID NO:1 and SEQ ID NO:2, complements thereof (e.g., one or more complements of SEQ ID NO:1 or SEQ ID NO:2) and any combinations thereof; and
(b) at least one reverse primer having a sequence selected from the group consisting of: SEQ ID NO:3, SEQ ID NO:4, SEQ ID NO:5, SEQ ID NO:6, complements thereof (e.g., one or more complements of SEQ ID NO:3, SEQ ID NO:4, SEQ ID NO:5, or SEQ ID NO:6) and any combinations thereof.

In another embodiment, the present invention relates to one or more probes for detecting HCV in a test sample. The one or more probes can include a probe having a sequence comprising or consisting of any of the sequences shown below in Table 2, a complement of any of the sequences shown below in Table 2 and any combinations of the sequences shown below in Table 2 and/or their complements. For example, the one or more probes may be only a single probe listed below in Table 2 or only a single complement of one of the probes listed below in Table 2 (such as for example, SEQ ID NO:7 or SEQ ID NO:8 or complements of all the probes listed below in Table 2 (complements of SEQ ID NOS:7 and 8) or any combinations thereof and/or combinations of the complements of the probes listed below in Table 2 (such as, for example, (a) SEQ ID NO:7 and SEQ ID NO:8; (b) SEQ ID NO:7 and the complement of SEQ ID NO:8; (c) the complement of SEQ ID NO:7 and SEQ ID NO:8; and (d) the complement of SEQ ID NO:7 and the complement of SEQ ID NO:8).

TABLE 2
Candidate Probe Sequences.

| SEQ ID NO: | SEQUENCE (5′ to 3′) |
|---|---|
| 7 | cgg cta gct gtg aaa ggt c |
| 8 | cgg cta gct gtg aaa ggt ccg |

In another embodiment, the present invention relates to a primer and probe set for detecting HCV in a test sample containing one or more of the primers described above in Table 1 and one or more of the probes described above in Table 2. For example, the primer and probe set can comprise the following:
(a) at least one forward primer having a sequence of: SEQ ID NO:1 and SEQ ID NO:2 or complements thereof and at least one reverse primer having a sequence of: SEQ ID NO:3, SEQ ID NO:4, SEQ ID NO:5, SEQ ID NO:6, or complements thereof; and
(b) at least one probe having a sequence of: SEQ ID NO:7, SEQ ID NO:8, or a complement thereof.

In one embodiment, the primer and probe set comprises:
(a) one forward primer having a sequence of: SEQ ID NO:1 or complements thereof and one reverse primer having a sequence of: SEQ ID NO:3, or complements thereof; and
(b) one probes having a sequence of: SEQ ID NO:8, or complements thereof. One or more oligonucleotide analogues can be prepared based on the primers and probes of the present invention. Such analogues may contain alternative structures such as peptide nucleic acids or “PNAs” (e.g, molecules with a peptide-like backbone instead of the phosphate sugar backbone of naturally occurring nucleic acids) and the like. These alternative structures, are also encompassed by the present invention. Similarly, it is understood that the primers and probes of the present invention may contain deletions, additions and/or substitutions of nucleic acid bases, to the extent that such alterations do not negatively affect the properties of these sequences. In particular, the alterations should not result in a significant decrease of the hybridizing properties of the primers and probes described herein.

The primers and probes of the present invention may be prepared by any of a variety of methods known in the art (See, for example, Sambrook et al., “Molecular Cloning. A Laboratory Manual,” 1989, 2. Supp. Ed., Cold Spring Harbour Laboratory Press: New York, N.Y.; “PCR Protocols. A Guide to Methods and Applications,” 1990, M. A. Innis (Ed.), Academic Press: New York, N.Y.; P. Tijssen “Hybridization with Nucleic Acid Probes—Laboratory Techniques in Biochemistry and Molecular Biology (Parts I and II),” 1993, Elsevier Science; “PCR Strategies,” 1995, M. A. Innis (Ed.), Academic Press: New York, N.Y.; and “Short Protocols in Molecular Biology,” 2002, F. M. Ausubel (Ed.), 5. Supp. Ed., John Wiley & Sons: Secaucus, N.J.). For example, primers and probes described herein may be prepared by chemical synthesis and polymerization based on a template as described, for example, in Narang et al., Meth. Enzymol., 1979, 68: 90-98; Brown et al., Meth. Enzymol., 1979, 68: 109-151 and Belousov et al., Nucleic Acids Res., 1997, 25: 3440-3444).

Syntheses may be performed on oligo synthesizers, such as those commercially available from Perkin Elmer/Applied Biosystems, Inc. (Foster City, Calif.), DuPont (Wilmington, Del.) or Milligen (Bedford, Mass.). Alternatively, the primers and probes of the present invention may be custom made and ordered from a variety of commercial sources well-known in the art, including, for example, the Midland Certified Reagent Company (Midland, Tex.), ExpressGen, Inc. (Chicago, Ill.), Operon Technologies, Inc. (Huntsville, Ala.), BioSearch Technologies, Inc. (Novato, Calif.), and many others.

Purification of the primers and probes of the present invention, where necessary or desired, may be carried out by any of a variety of methods well-known in the art. Purification of primers and probes can be performed either by native acrylamide gel electrophoresis, by anion-exchange HPLC as described, for example, by Pearson et al., J. Chrom., 1983, 255: 137-149 or by reverse phase HPLC (See, McFarland et al., Nucleic Acids Res., 1979, 7: 1067-1080).

As previously mentioned, modified primers and probes may be prepared using any of several means known in the art. Non-limiting examples of such modifications include methylation, substitution of one or more of the naturally occurring nucleotides with an analog, and internucleotide modifications such as, for example, those with uncharged linkages (e.g., methyl phosphonates, phosphotriesters, phosphoramidates, carbamates, etc), or charged linkages (e.g., phosphorothioates, phosphorodithioates, etc). Primers and probes may contain one or more additional covalently linked moieties, such as, for example, proteins (e.g., nucleases, toxins, antibodies, signal peptides, poly-L-lysine, etc), intercalators (e.g., acridine, psoralen, etc), chelators (e.g., to chelate metals, radioactive metals, oxidative metals, etc), and alkylators. Primers and probes may also be derivatized by formation of a methyl or ethyl phosphotriester or an alkyl phosphoramidate linkage. Furthermore, primers and/or probes of the present invention may be modified with a detectable label.

As alluded to above, in certain embodiments of the present invention, the primers and/or the probes may be labeled with a detectable label or moiety before being used in one or more amplification/detection methods. Preferably, for use in the methods described herein, one or more probes are labeled with a detectable label or moiety. The role of a detectable label is to allow visualization and/or detection of amplified target sequences (e.g., amplicons). Preferably, the detectable label is selected such that it generates a signal which can be measured and whose intensity is related (e.g., proportionally) to the amount of amplification product in the test sample being analyzed.

The association between one or more labeled probes and the detectable label can be covalent or non-covalent. Labeled probes can be prepared by incorporation of, or conjugation to, a detectable moiety. Labels can be attached directly to the nucleic acid sequence or indirectly (e.g., through a linker). Linkers or spacer arms of various lengths are known in the art and are commercially available, and can be selected to reduce steric hindrance, or to confer other useful or desired properties to the resulting labeled molecules (See, for example, Mansfield et al., Mol. Cell. Probes, 1995, 9: 145-156).

Methods for labeling oligonucleotides, such as primers and/or probes, are well-known to those skilled in the art. Reviews of labeling protocols and label detection techniques can be found in, for example, L. J. Kricka, Ann. Clin. Biochem., 2002, 39: 114-129; van Gijlswijk et al., Expert Rev. Mol. Diagn., 2001, 1: 81-91; and Joos et al., J. Biotechnol., 1994, 35: 135-153. Standard nucleic acid labeling methods include: incorporation of radioactive agents, direct attachments of fluorescent dyes (See, Smith et al., Nucl. Acids Res., 1985, 13: 2399-2412) or enzymes (See, Connoly et al., Nucl. Acids. Res., 1985, 13: 4485-4502); chemical modifications of nucleic acid molecules rendering them detectable immunochemically or by other affinity reactions (See, Broker et al., Nucl. Acids Res., 1978, 5: 363-384; Bayer et al., Methods of Biochem. Analysis, 1980, 26: 1-45; Langer et al., Proc. Natl. Acad. Sci. USA, 1981, 78: 6633-6637; Richardson et al., Nucl. Acids Res., 1983, 11: 6167-6184; Brigati et al., Virol., 1983, 126: 32-50; Tchen et al., Proc. Natl. Acad. Sci. USA, 1984, 81: 3466-3470; Landegent et al., Exp. Cell Res., 1984, 15: 61-72; and A. H. Hopman et al., Exp. Cell Res., 1987, 169: 357-368); and enzyme-mediated labeling methods, such as random priming, nick translation, PCR, and tailing with terminal transferase (For a review on enzymatic labeling, see, for example, Temsamani et al., Mol. Biotechnol., 1996, 5: 223-232).

Any of a wide variety of detectable labels can be used in the present invention. Suitable detectable labels include, but are not limited to, various ligands, radionuclides or radioisotopes (e.g., ³²P, ³⁵S, ³H, ¹⁴C, ¹²⁵I, ¹³¹I, and the like); fluorescent dyes; chemiluminescent agents (e.g., acridinium esters, stabilized dioxetanes, and the like); spectrally resolvable inorganic fluorescent semiconductor nanocrystals (e.g., quantum dots), metal nanoparticles (e.g., gold, silver, copper and platinum) or nanoclusters; enzymes (e.g., horseradish peroxidase, beta-galactosidase, luciferase, alkaline phosphatase); colorimetric labels (e.g., dyes, colloidal gold, and the like); magnetic labels (e.g., Dynabeads™); and biotin and dioxigenin, or other haptens and proteins for antisera or monoclonal antibodies are available.

In certain embodiments, the contemplated probes are fluorescently labeled. Numerous known fluorescent labeling moieties of a wide variety of chemical structures and physical characteristics are suitable for use in the practice of this invention. Suitable fluorescent dyes include, but are not limited to, Quasar® dyes available from Biosearch Technologies, Novato, Calif.), fluorescein and fluorescein dyes (e.g., fluorescein isothiocyanine (FITC), naphthofluorescein, 4′,5′-dichloro-2′,7′-dimethoxy-fluorescein, 6-carboxyfluoresceins (e.g., FAM), VIC, NED, carbocyanine, merocyanine, styryl dyes, oxonol dyes, phycoerythrin, erythrosin, eosin, rhodamine dyes (e.g., carboxytetramethylrhodamine or TAMRA, carboxyrhodamine 6G, carboxy-X-rhodamine (ROX), lissamine rhodamine B, rhodamine 6G, rhodamine Green, rhodamine Red, tetramethylrhodamine or TMR), coumarin and coumarin dyes (e.g., methoxycoumarin, dialkylaminocoumarin, hydroxycoumarin and aminomethylcoumarin or AMCA), Oregon Green Dyes (e.g., Oregon Green 488, Oregon Green 500, Oregon Green 514), Texas Red, Texas Red-X, Spectrum Red™, Spectrum Green™, cyanine dyes (e.g., Cy-3™, Cy-5™, Cy-3.5™, Cy-5.5™), Alexa Fluor dyes (e.g., Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), IRDyes (e.g., IRD40, IRD 700, IRD 800), and the like. Examples of other suitable fluorescent dyes that can be used and methods for linking or incorporating fluorescent dyes to oligonucleotides, such as probes, can be found in RP Haugland, “The Handbook of Fluorescent Probes and Research Chemicals”, Publisher, Molecular Probes, Inc., Eugene, Oreg. (June 1992)). Fluorescent dyes, as well as labeling kits, are commercially available from, for example, Amersham Biosciences, Inc. (Piscataway, N.J.), Molecular Probes Inc. (Eugene, Oreg.), and New England Biolabs Inc. (Beverly, Mass.).

Rather than being directly detectable themselves, some fluorescent groups (donors) transfer energy to another fluorescent group (acceptor) in a process of fluorescence resonance energy transfer (FRET), and the second group produces the detectable fluorescent signal. In these embodiments, the probe may, for example, become detectable when hybridized to an amplified target sequence. Examples of FRET acceptor/donor pairs suitable for use in the present invention include, for example, fluorescein/tetramethylrhodamine, IAEDANS/FITC, IAEDANS/5-(iodoacetomido)fluorescein, B-phycoerythrin/Cy-5, and EDANS/Dabcyl, among others.

FRET pairs also include the use of physically-linked fluorescent reporter/quencher pairs. For example, a detectable label and a quencher moiety may be individually attached to either the 5′ end or the 3′ end of a probe, therefore placing the detectable label and the quencher moiety at opposite ends of the probe, or apart from one another along the length of the probe. During such time as the probe is not bound to its target sequence, the detectable label and quencher moiety are reversibly maintained within such proximity that the quencher blocks the detection of the detectable label. Upon binding of the probe to a target sequence, the detectable label and quencher moiety are separated thus permitting detection of the detectable label under appropriate conditions.

The use of such systems in TaqMan® assays (as described, for example, in U.S. Pat. Nos. 5,210,015, 5,804,375, 5,487,792, and 6,214,979) or as Molecular Beacons (as described, for example in, Tyagi et al., Nature Biotechnol., 1996, 14: 303-308; Tyagi et al., Nature Biotechnol., 1998, 16: 49-53; Kostrikis et al., Science, 1998, 279: 1228-1229; Sokol et al., Proc. Natl. Acad. Sci. USA, 1998, 95: 11538-11543; Marras et al., Genet. Anal., 1999, 14: 151-156; and U.S. Pat. Nos. 5,846,726, 5,925,517, 6,277,581 and 6,235,504) is well-known to those skilled in the art. With the TaqMan® assay format, products of the amplification reaction can be detected as they are formed in a “real-time” manner: amplification product/probe hybrids are formed and detected while the reaction mixture is under amplification conditions.

In some embodiments of the present invention, the PCR detection probes are TaqMan®-like probes that are labeled at the 5′-end with a fluorescent moiety and at the 3′-end with a quencher moiety or alternatively the fluorescent moiety and quencher moiety are in reverse order, or further they may be placed along the length of the sequence to provide adequate separation when the probe hybridizes to a target sequence to allow satisfactory detection of the fluorescent moiety. Suitable fluorophores and quenchers for use with TaqMan®-like probes are disclosed in U.S. Pat. Nos. 5,210,015, 5,804,375, 5,487,792, and 6,214,979, and WO 01/86001. Examples of quenchers include, but are not limited, to DABCYL (e.g., 4-(4′-dimethylaminophenylazo)-benzoic acid) succinimidyl ester, diarylrhodamine carboxylic acid, succinimidyl ester (or QSY-7), and 4′,5′-dinitrofluorescein carboxylic acid, succinimidyl ester (or QSY-33) (all of which are available from Molecular Probes (which is part of Invitrogen, Carlsbad, Calif.)), quencher1 (Q1; available from Epoch Biosciences, Bothell, Wash.), or “Black hole quenchers” BHQ-1, BHQ-2, and BHQ-3 (available from BioSearch Technologies, Inc., Novato, Calif.). In certain embodiments, the PCR detection probes are TaqMan®-like probes that are labeled at the 5′ end with FAM and at the 3′ end with a Black Hole Quencher® or Black Hole Quencher® plus (Biosearch Technologies, Novato, Calif.).

A “tail” of normal or modified nucleotides can also be added to probes for detectability purposes. A second hybridization with nucleic acid complementary to the tail and containing one or more detectable labels (such as, for example, fluorophores, enzymes, or bases that have been radioactively labeled) allows visualization of the amplicon/probe hybrids.

The selection of a particular labeling technique may depend on the situation and may be governed by several factors, such as the ease and cost of the labeling method, spectral spacing between different detectable labels used, the quality of sample labeling desired, the effects of the detectable moiety on the hybridization reaction (e.g., on the rate and/or efficiency of the hybridization process), the nature of the amplification method used, the nature of the detection system, the nature and intensity of the signal generated by the detectable label, and the like.

### C. AMPLIFICATION METHODS

The use of primers or primer sets of the present invention to amplify HCV target sequences in test samples is not limited to any particular nucleic acid amplification technique or any particular modification thereof. In fact, the primers and primer sets of the present invention can be employed in any of a variety of nucleic acid amplification methods that are known in the art (See, for example, Kimmel et al., Methods Enzymol., 1987, 152: 307-316; Sambrook et al., “Molecular Cloning. A Laboratory Manual”, 1989, 2.Supp. Ed., Cold Spring Harbour Laboratory Press: New York, N.Y.; “Short Protocols in Molecular Biology”, F. M. Ausubel (Ed.), 2002, 5. Supp. Ed., John Wiley & Sons: Secaucus, N.J.).

Such nucleic acid amplification methods include, but are not limited to, the Polymerase Chain Reaction (PCR). PCR is described in a number of references, such as, but not limited to, “PCR Protocols: A Guide to Methods and Applications”, M. A. Innis (Ed.), 1990, Academic Press: New York; “PCR Strategies”, M. A. Innis (Ed.), 1995, Academic Press: New York; “Polymerase chain reaction: basic principles and automation in PCR. A Practical Approach”, McPherson et al. (Eds.), 1991, IRL Press: Oxford; Saiki et al., Nature, 1986, 324: 163; and U.S. Pat. Nos. 4,683,195, 4,683,202 and 4,889,818. Variations of PCR including, TaqMan®-based assays (See, Holland et al., Proc. Natl. Acad. Sci., 1991, 88: 7276-7280), and reverse transcriptase polymerase chain reaction (or RT-PCR, described in, for example, U.S. Pat. Nos. 5,322,770 and 5,310,652) are also included.

Generally, in PCR, a pair of primers is added to a test sample obtained from a subject (and thus contacted with the test sample) in excess to hybridize to the complementary strands of the target nucleic acid. The primers are each extended by a DNA polymerase using the target sequence as a template. The extension products become targets themselves after dissociation (denaturation) from the original target strand. New primers are then hybridized and extended by the polymerase, and the cycle is repeated to exponentially increase the number of amplicons. Examples of DNA polymerases capable of producing primer extension products in PCR reactions include, but are not limited to, E. coli DNA polymerase I, Klenow fragment of DNA polymerase I, T4 DNA polymerase, thermostable DNA polymerases isolated from Thermus aquaticus (Taq), available from a variety of sources (e.g., Perkin Elmer, Waltham, Mass.), Thermus thermophilus (USB Corporation, Cleveland, Ohio), Bacillus stereothermophilus (Bio-Rad Laboratories, Hercules, Calif.), AmpliTaq Gold® Enzyme (Applied Biosystems, Foster City, Calif.), recombinant Thermus thermophilus (rTth) DNA polymerase (Applied Biosystems, Foster City, Calif.) or Thermococcus litoralis (“Vent” polymerase, New England Biolabs, Ipswich, Mass.). RNA target sequences may be amplified by first reverse transcribing (RT) the mRNA into cDNA, and then performing PCR (RT-PCR), as described above. Alternatively, a single enzyme may be used for both steps as described in U.S. Pat. No. 5,322,770.

In addition to the enzymatic thermal amplification methods described above, isothermal enzymatic amplification reactions can be employed to amplify HCV sequences using primers and primer sets of the present invention (Andras et al., Mol. Biotechnol., 2001, 19: 29-44). These methods include, but are not limited to, Transcription-Mediated Amplification (TMA; TMA is described in Kwoh et al., Proc. Natl. Acad. Sci. USA, 1989, 86: 1173-1177; Giachetti et al., J. Clin. Microbiol., 2002, 40: 2408-2419; and U.S. Pat. No. 5,399,491); Self-Sustained Sequence Replication (3SR; 3SR is described in Guatelli et al., Proc. Natl. Acad. Sci. USA, 1990, 87: 1874-1848; and Fahy et al., PCR Methods and Applications, 1991, 1: 25-33); Nucleic Acid Sequence Based Amplification (NASBA; NASBA is described in, Kievits et al., J. Virol. Methods, 1991, 35: 273-286; and U.S. Pat. No. 5,130,238) and Strand Displacement Amplification (SDA; SDA is described in Walker et al., PNAS, 1992, 89: 392-396; EP 0 500 224 A2).

### D. DETECTION METHODS

In certain embodiments of the present invention, the probes described herein are used to detect amplification products generated by the amplification reaction. The probes described herein may be employed using a variety of well-known homogeneous or heterogeneous methodologies.

Homogeneous detection methods include, but are not limited to, the use of FRET labels that are attached to the probes and that emit a signal in the presence of the target sequence, Molecular Beacons (See, Tyagi et al., Nature Biotechnol., 1996, 14: 303-308; Tyagi et al., Nature Biotechnol., 1998, 16: 49-53; Kostrikis et al., Science, 1998, 279: 1228-1229; Sokol et al., Proc. Natl. Acad. Sci. USA, 1998, 95: 11538-11543; Marras et al., Genet. Anal., 1999, 14: 151-156; and U.S. Pat. Nos. 5,846,726, 5,925,517, 6,277,581 and 6,235,504), and the TaqMan® assays (See, U.S. Pat. Nos. 5,210,015; 5,804,375; 5,487,792 and 6,214,979 and WO 01/86001). Using these detection techniques, products of the amplification reaction can be detected as they are formed, namely, in a real time manner. As a result, amplification product/probe hybrids are formed and detected while the reaction mixture is under amplification conditions.

In certain embodiments, the probes of the present invention are used in a TaqMan® assay. In a TaqMan® assay, analysis is performed in conjunction with thermal cycling by monitoring the generation of fluorescence signals. The assay system has the capability of generating quantitative data allowing the determination of target copy numbers. For example, standard curves can be generated using serial dilutions of previously quantified suspensions of one or more HCV sequences, against which unknown samples can be compared. The TaqMan® assay is conveniently performed using, for example, AmpliTaq Gold™ DNA polymerase, which has endogenous 5′ nuclease activity, to digest a probe labeled with both a fluorescent reporter dye and a quencher moiety, as described above. Assay results are obtained by measuring changes in fluorescence that occur during the amplification cycle as the probe is digested, uncoupling the fluorescent and quencher moieties and causing an increase in the fluorescence signal that is proportional to the amplification of the target sequence.

Other examples of homogeneous detection methods include hybridization protection assays (HPA). In such assays, the probes are labeled with acridinium ester (AE), a highly chemiluminescent molecule (See, Weeks et al., Clin. Chem., 1983, 29: 1474-1479; Berry et al., Clin. Chem., 1988, 34: 2087-2090), using a non-nucleotide-based linker arm chemistry (See, U.S. Pat. Nos. 5,585,481 and 5,185,439). Chemiluminescence is triggered by AE hydrolysis with alkaline hydrogen peroxide, which yields an excited N-methyl acridone that subsequently deactivates with emission of a photon. In the absence of a target sequence, AE hydrolysis is rapid. However, the rate of AE hydrolysis is greatly reduced when the probe is bound to the target sequence. Thus, hybridized and un-hybridized AE-labeled probes can be detected directly in solution without the need for physical separation.

Heterogeneous detection systems are also well-known in the art and generally employ a capture agent to separate amplified sequences from other materials in the reaction mixture. Capture agents typically comprise a solid support material (e.g., microtiter wells, beads, chips, and the like) coated with one or more specific binding sequences. A binding sequence may be complementary to a tail sequence added to oligonucleotide probes of the invention. Alternatively, a binding sequence may be complementary to a sequence of a capture oligonucleotide, itself comprising a sequence complementary to a tail sequence of a probe. After separation of the amplification product/probe hybrids bound to the capture agents from the remaining reaction mixture, the amplification product/probe hybrids can be detected using any detection methods, such as those described herein.

### E. DETECTING HCV IN TEST SAMPLES

The present invention provides methods for detecting the presence of HCV in a test sample. Further, HCV levels may be quantified per test sample by comparing test sample detection values against standard curves generated using serial dilutions of previously quantified suspensions of one or more HCV sequences or other standardized HCV profiles.

Typically, methods of the invention first involve obtaining a test sample from a subject. Contemplated subjects include any mammals such as dogs, cats, rabbits, mice, rats, goats, sheep, cows, pigs, horses, non-human primates, and preferably humans. The test sample can be obtained from the subject using routine techniques known to those skilled in the art. Preferably, the test sample contains or is suspected of containing at least one HCV genotype.

After the test sample is obtained from a subject, the test sample is contacted with primers (and optionally one or more probes) from at least one of the primer sets or primer and probe sets disclosed herein to form a reaction mixture. The reaction mixture is then placed under amplification conditions. The primers hybridize to complementary HCV nucleic acids in the test sample. The primer hybridized HCV nucleic acid in the sample is amplified and at least one amplification product (namely, at least one target sequence) is generated.

At least one amplification product is detected by detecting the hybridization between at least one amplification product and at least one of the probes of the present invention (such as one or more probes from the primer and probe sets described herein). Specifically, detection of at least one amplification product with one or more of the probes having a sequence of SEQ ID NO:7, SEQ ID NO:8, or a complement thereof indicates the presence of at least one HCV genotype in the test sample.

### F. KITS

In another embodiment, the present invention provides kits including materials and reagents useful for the detection of HCV according to methods described herein. The kits can be used by diagnostic laboratories, experimental laboratories, or practitioners. In certain embodiments, the kits comprise at least one of the primer sets or primer and probe sets described in Section B herein and optionally, amplification reagents. Each kit preferably comprises amplification reagents for a specific amplification method. Thus, a kit adapted for use with NASBA preferably contains primers with an RNA polymerase promoter linked to the target binding sequence, while a kit adapted for use with SDA preferably contains primers including a restriction endonuclease recognition site 5′ to the target binding sequence. Similarly, when the kit is adapted for use in a 5′ nuclease assay, such as the TaqMan® assay, the probes of the present invention can contain at least one fluorescent reporter moiety and at least one quencher moiety.

Suitable amplification reagents additionally include, for example, one or more of: buffers, reagents, enzymes having reverse transcriptase and/or polymerase activity or exonuclease activity, enzyme cofactors such as magnesium or manganese; salts; deoxynucleotide triphosphates (dNTPs) suitable for carrying out the amplification reaction.

Depending on the procedure, kits may further comprise one or more of: wash buffers, hybridization buffers, labeling buffers, detection means, and other reagents. The buffers and/or reagents are preferably optimized for the particular amplification/detection technique for which the kit is intended. Protocols for using these buffers and reagents for performing different steps of the procedure may also be included in the kit.

Furthermore, kits may be provided with an internal control as a check on the amplification efficiency, to prevent occurrence of false negative test results due to failures in the amplification, to check on cell adequacy, sample extraction, etc. An optimal internal control sequence is selected in such a way that it will not compete with the target nucleic acid sequence in the amplification reaction. Such internal control sequences are known in the art.

Kits may also contain reagents for the isolation of nucleic acids from test samples prior to amplification before nucleic acid extraction.

The reagents may be supplied in a solid (e.g., lyophilized) or liquid form. Kits of the present invention may optionally comprise different containers (e.g., vial, ampoule, test tube, flask, or bottle) for each individual buffer and/or reagent. Each component will generally be suitable as aliquoted in its respective container or provided in a concentrated form. Other containers suitable for conducting certain steps of the amplification/detection assay may also be provided. The individual containers are preferably maintained in close confinement for commercial sale.

Kits may also comprise instructions for using the amplification reagents and primer sets or primer and probe described herein: for processing the test sample, extracting nucleic acid molecules, and/or performing the test; and for interpreting the results obtained as well as a notice in the form prescribed by a governmental agency. Such instructions optionally may be in printed form or on CD, DVD, or other format of recorded media.

By way of example, and not of limitation, examples of the present disclosures shall now be given.

### Example 1

### Materials and Methods

### A. Design of HCV Primers and Probes

All oligonucleotides used in the Examples were synthesized using standard oligonucleotide synthesis methodology known to those skilled in the art. All of the probes are single-stranded oligonucleotides labeled using routine techniques known in the art, with a fluorophore at the 5′ end and a quenching moiety at the 3′ end. For example, for SEQ ID NO:8, the 5′ label is FAM and the 3′ label is Black Hole Quencher (BHQ), such as BHQ1-dT. The primers (SEQ ID NO:1 and SEQ ID NO:3) are unlabeled.

### B. Real-Time PCR

HCV RNA was extracted, concentrated and purified from samples using magnetic micro-particle technology that captures nucleic acids and washes the particles to remove unbound sample components (See, for example, U.S. Pat. No. 5,234,809). The bound nucleic acids were eluted and added directly to the PCR reaction mix. Reverse transcription and the real-time PCR reaction were performed in a single tube reaction. An HCV primer mix including SEQ ID NO:1 (forward) and SEQ ID NO:3 (reverse), collectively referred to herein as the “HCV Primer Mix,” was used to amplify HCV genome sequences. Signal for HCV 3′-UTR was generated with an HCV specific probe (SEQ ID NO:8). Besides the primers and probe, the PCR reaction consisted of: 10 Units rTth enzyme, 2.5 mM manganese chloride (as activation reagent) and other amplification reagents (containing 0.3 mM dNTPs, 15 nM ROX reference dye, 200 nM aptamer in Bicene buffer). Fifty microliters of eluted nucleic acids and fifty microliters of PCR reaction mix described above were combined in each well of a 96 well reaction plate and sealed with an optical adhesive cover. This plate was amplified as described below.

Real-time amplification/detection was carried out on an Abbott m2000rt instrument (Abbott Molecular Inc., Des Plaines, Ill.) using the following cycling conditions: 1 cycle at 95° C. for 45 seconds and 62° C. for 30 minutes; and 50 cycles at 95° C. 45 seconds and 60° C. 45 seconds. Fluorescence measurements were recorded during the read step (60° C.) of the 50 cycles.

### Example 2

### Sensitivity

To assess the relative sensitivity of the presently disclosed assay (“3′UTR Assay”) in relation to other HCV detection assays, limit of detection performance was evaluated using HCV targets of varied origin.

A. Sensitivity with Synthetic RNA Constructs.

A study comparing the limits of detection of the RealTime HCV and the 3′UTR (using the HCV Primer Mix and Real-Time PCR of Example 1) assays used an in vitro RNA construct target, containing both the 5′UTR and 3′UTR sequences, which was obtained from Apath (St. Louis, Mo.). In Table 3 (below) both the 3′UTR and RealTime assays demonstrated 3 of 3 hits at 153.77 IU/ml, 3 of 3 hits and 1 of 3 hits at 2.97 IU/ml, respectively, and 1 of 3 hits each at 2.00 IU/ml.

TABLE 4
Comparison of Hit Rates for 3′UTR and RealTime HCV (5′UTR)
Assays for Synthetic RNA Constructs.

Hit rate

| Target level | 3′UTR | RealTime |
|---|---|---|
| (IU/ml) | IVT | IVT |
| 44616.44 | 3/3 | 3/3 |
| 3695.53 | 3/3 | 3/3 |
| 153.77 | 3/3 | 3/3 |
| 2.97 | 3/3 | 1/3 |
| 2.00 | 1/3 | 1/3 |

The data from Table 3 correspond to predicted limits of detection of 3.31 IU/ml for RealTime HCV and 2.05 IU/ml for the presently disclosed 3′UTR HCV assay using the synthetic RNA construct target (See, Table 4 below).

TABLE 4
Probit Results - 3′UTR and RealTime HCV (5′UTR)
Assays for Synthetic RNA Constructs.

| | Predicted Target |
|---|---|
| | LOD (IU/ml) |
| 3′UTR | 2.05 |
| RealTime HCV | 3.31 |

The above data of Tables 3 and 4 demonstrate the currently disclosed 3′UTR assay has equivalent sensitivity to the RealTime HCV assay (which targets the 5′UTR). Therefore, these data demonstrate that the currently disclosed 3′UTR assay provides an alternate assay for detecting HCV with equal sensitivity to a 5′UTR assay, but by targeting the 3′UTR.

B. Sensitivity to Specimen-Derived HCV.

A high titer HCV viral eluate having genotype 3 from a patient specimen was obtained from ProMedDx (Norton, Mass.) was serially diluted and quantitated by the RealTime HCV assay (using the HCV Primer Mix and Real-Time PCR of Example 1). This dilution series was used to compare the limit of detection performance of the presently disclosed 3′UTR assay to that of the RealTime HCV assay.

TABLE 5
Comparison of Hit Rates for the 3′UTR and RealTime HCV (5′UTR)
Assays for Specimen-derived HCV.

| Target level | 3′UTR | 5′UTR |
|---|---|---|
| IU/ml | Viral eluate | Viral eluate |
| 80345.98 | 3/3 | 3/3 |
| 8435.16 | 3/3 | 3/3 |
| 778.86 | 3/3 | 3/3 |
| 89.83 | 3/3 | 3/3 |
| 8.99 | 1/3 | 3/3 |
| 2.50 | 0/3 | 3/3 |
| Negative | 0/3 | 0/3 |

The data from Table 5 corresponds to predicted limits of detection of 1.26 IU/ml for the RealTime HCV assay and 9.26 IU/ml for the presently disclosed 3′UTR HCV assay using the specimen-derived HCV (See, Table 6 below).

TABLE 6
Probit Results - IVT for the 3′UTR and RealTime HCV (5′UTR)
Assays for Specimen-derived HCV.

| | Predicted Target |
|---|---|
| | LOD (IU/ml) |
| 3′UTR | 9.26 |
| RealTime HCV | 1.26 |

The above data in Tables 5 and 6, consistent with those disclosed above, demonstrate comparable sensitivity for the presently disclosed 3′UTR assay compared to the RealTime HCV assay (which targets the 5′UTR).

### Example 3

### Multiple Genotype Detection

To determine the ability of the presently disclosed 3′UTR assay to detect the different HCV genotypes, thirty-two (32) patient specimens from Teragenix (Ft. Lauderdale, Fla.) representing HCV genotypes 1 through 6 were prepared and quantitated using the 3′UTR (using the HCV Primer Mix and Real-Time PCR of Example 1) assay. All specimen results were compared to previously generated RealTime assay (5′UTR) results (Table 7).

TABLE 7
Comparison of Quantitation of 3′UTR and RealTime HCV Assays
for HCV Genotype Detection.

| HCV | | | RealTime HCV |
|---|---|---|---|
| Genotype | 3′UTR | 3′UTR Quantitation | Quantitation |
| Samples | Threshold cycle | log IU/ml | log IU/ml |
| 1a-2 | 24.76 | 5.37 | 5.39 |
| 1a-3 | 25.06 | 5.29 | 5.22 |
| 1a-4 | 21.73 | 6.11 | 6.35 |
| 1a-5 | 21.92 | 6.07 | 6.23 |
| 1b-1 | 24.42 | 5.45 | 5.19 |
| 1b-2 | 22.81 | 5.85 | 5.97 |
| 1b-3 | 22.71 | 5.87 | 5.65 |
| 1b-4 | 24.8 | 5.36 | 5.08 |
| 2a-3 | 26.83 | 4.86 | 4.61 |
| 2a-4 | 20.38 | 6.44 | 6.64 |
| 2b-1 | 23.08 | 5.78 | 5.83 |
| 2b-6 | 21.93 | 6.06 | 6.15 |
| 2b-7 | 26.6 | 4.92 | 4.67 |
| 2b-9 | 21.9 | 6.07 | 6.10 |
| 2b-10 | 20.33 | 6.46 | 6.75 |
| 2b-11 | 20.09 | 6.52 | 6.73 |
| 3-3 | 27.71 | 4.64 | 5.46 |
| 3-10 | 29.86 | 4.12 | 4.73 |
| 3-14 | 26.39 | 4.97 | 5.75 |
| 3-16 | 28.12 | 4.54 | 5.35 |
| 4-6 | 24.5 | 5.43 | 5.08 |
| 4-7 | 27.72 | 4.64 | 4.93 |
| 4-8 | 29.13 | 4.29 | 3.80 |
| 4-9 | 24.12 | 5.53 | 5.41 |
| 5-1 | 21.96 | 6.06 | 5.95 |
| 5-2 | 24.38 | 5.46 | 5.25 |
| 5-3 | 25.05 | 5.30 | 4.89 |
| 5-4 | 24.38 | 5.46 | 5.09 |
| 6-1 | 22.61 | 5.90 | 6.20 |
| 6-2 | 22.32 | 5.97 | 6.25 |
| 6-3 | 23.06 | 5.79 | 6.08 |
| 6-4 | 20.29 | 6.47 | 6.74 |

The results in Table 7 demonstrate the ability of the presently disclosed 3′UTR assay to detect HCV genotypes 1 through 6. Further, the sensitivity of the presently disclosed 3′UTR assay for each genotype is comparable to that of the RealTime HCV assay.

### Example 4

### Alternate HCV Primer Mix—Multiple Reverse Primers

The following Example used a HCV primer mix containing SEQ ID NO:1 (forward), SEQ ID NO:3 (reverse) and SEQ ID NO:6 (reverse) to amplify HCV genome sequences. Signal for HCV 3′-UTR was generated with an HCV specific probe (SEQ ID NO:7). PCR reaction and cycling conditions previously described apply to this example. Dilutions of two high titer specimens, one genotype 1a and one genotype 3, were tested with both the presently disclosed 3′UTR assay and the RealTime HCV assay.

TABLE 8
Multiple Reverse Primer Assay Results.

| HCV Genotype and | 3′UTR | 3′UTR Mean | 5′UTR |
|---|---|---|---|
| target concentration | Mean Log IU/ml | HCV Ct | Mean Log IU/m |
| GT 1 log 3 IU/ml | 3.96 | 31.03 | 2.58 |
| GT 3 log 3 IU/ml | 3.47 | 32.73 | 2.78 |
| GT 3 log 5 IU/ml | 5.45 | 26.24 | 4.85 |

The addition of the second reverse primer (SEQ ID NO:6) partially compensates for the Threshold cycle delay seen with HCV genotype 3 three specimens (observed in Table 8) to enhance the sensitivity of the presently disclosed 3′UTR assay. The enhanced sensitivity observed is believed to be achieved through increased amplification of target sequence via the second reverse primer (SEQ ID NO:6) which compensates for a mismatch with SEQ ID NO:3.

One skilled in the art would readily appreciate that the present invention is well adapted to carry out the objects and obtain the ends and advantages mentioned, as well as those inherent therein. The molecular complexes and the methods, procedures, treatments, molecules, and specific compounds described herein are presently representative of preferred embodiments, are exemplary, and are not intended as limitations on the scope of the invention. It will be readily apparent to one skilled in the art that various substitutions and modifications may be made to the invention as disclosed herein without departing from the scope and spirit of the invention.

All patents and publications mentioned in the specification are indicative of the levels of those skilled in the art to which the invention pertains. All patents and publications are herein incorporated by reference.

The invention illustratively described herein may suitably be practiced in the absence of any element or elements, limitation or limitations which is not specifically disclosed herein. The terms and expressions which have been employed are used as terms of description and not of limitation and there is no intention in the use of such terms and expressions of excluding any equivalents of the features shown and described or portions thereof. It is recognized that various modifications are possible within the scope of the invention claimed. Thus, it should be understood that although the present invention has been specifically disclosed by preferred embodiments, optional features, modifications and variations of the concepts herein disclosed may be resorted to by one skilled in the art and such modifications and variations are considered to be within the scope of this invention as defined by the appended claims.

What is claimed is:

1. A primer for amplifying Hepatitis C virus in a test sample, wherein the primer has a sequence selected from the group consisting of: SEQ ID NO:1, SEQ ID NO:2, SEQ ID NO:3, SEQ ID NO:4, SEQ ID NO:5, SEQ ID NO:6, and complements thereof.

2. A probe for detecting Hepatitis C virus in a test sample, wherein the probe has a sequence selected from the group consisting of: SEQ ID NO:7, SEQ ID NO:8, and complements thereof.

3. A primer set for amplifying Hepatitis C virus in a test sample, the primer set comprising:
(a) at least one forward primer having a sequence selected from the group consisting of: SEQ ID NO:1, SEQ ID NO:2, complements thereof, and any combinations thereof; and
(b) at least one reverse primer having a sequence selected from the group consisting of: SEQ ID NO:3, SEQ ID NO:4, SEQ ID NO:5, SEQ ID NO:6, complements thereof, and any combinations thereof.

4. A primer and probe set for detecting Hepatitis C virus in a test sample, comprising:
(a) two forward primers having a sequence of: SEQ ID NO:1 and SEQ ID NO:2, or complements thereof, and four reverse primers having a sequence of: SEQ ID NO:3, SEQ ID NO:4, SEQ ID NO:5, and SEQ ID NO:6, or complements thereof; and
(b) two probes having a sequence of: SEQ ID NO:7 and SEQ ID NO:8, or complements thereof.

5. A method for detecting Hepatitis C virus in a test sample, the method comprising the steps of:
(a) contacting a test sample with at least one forward primer having a sequence selected from the group consisting of SEQ ID NO:1 and SEQ ID NO:2, or complements thereof and at least one reverse primer having a sequence selected from the group consisting of: SEQ ID NO:3, SEQ ID NO:4, SEQ ID NO:5, and SEQ ID NO:6 or complements thereof under amplification conditions to generate a first target sequence; and
(b) detecting hybridization between the first target sequence and at least one probe as an indication of the presence of Hepatitis C virus in the test sample, wherein the at least one probe has a sequence selected from the group consisting of: SEQ ID NO:7 or SEQ ID NO:8, or complements thereof.

6. The method of claim 5, wherein the amplification conditions comprise submitting the test sample to an amplification reaction carried out in the presence of suitable amplification reagents.

7. The method of claim 6, wherein the amplification reaction comprises at least one of:
a) PCR;
b) real-time PCR; or
c) reverse-Transcriptase PCR (RT-PCR).

8. The method of claim 5, wherein, the at least one probe is labeled with a detectable label.

9. The method of claim 8, wherein the detectable label is directly attached to the at least one probe.

10. The method of claim 8, wherein the detectable label is indirectly attached to the at least one probe.

11. The method of claim 8, wherein the detectable label is directly detectable.

12. The method of claim 8, wherein the detectable label is indirectly detectable.

13. The method of claim 8, wherein the detectable label comprises a fluorescent moiety attached at a 5′ end of the at least one probe.

14. The method of claim 13, wherein the at least one probe further comprises a quencher moiety attached at a 3′ end of the at least one probe.

15. The method of claim 5, further comprising the steps of:
(a) contacting the test sample with a forward primer having a sequence of SEQ ID NO:1 or a complement thereof and a reverse primer having a sequence of SEQ ID NO:3 or a complement thereof under amplification conditions to generate a second target sequence; and
(b) detecting hybridization between the second target sequence and a probe having a sequence of SEQ ID NO:7 or a complement thereof as an indication of the presence of HCV in the test sample.

16. A kit for detecting Hepatitis C virus in a test sample, the kit comprising:
(a) at least one forward primer having a sequence selected from the group consisting of: SEQ ID NO:1, SEQ ID NO:2, complements thereof, and any combinations thereof;
(b) at least one reverse primer having a sequence selected from the group consisting of: SEQ ID NO:3, SEQ ID NO:4, SEQ ID NO:5, SEQ ID NO:6, complements thereof, and any combinations thereof; and
(c) amplification reagents.

17. The kit of claim 16, further comprising at least one probe, wherein the at least one probe is selected from the group consisting of: SEQ ID NO:7, SEQ ID NO:8, and complements thereof.


SEQUENCE LISTING (8 sequences)

SEQ ID NO: 1 - DNA, 20 bp, Artificial Sequence
  Description of Artificial Sequence Synthetic primer
gctccatcttagccctagtc

SEQ ID NO: 2 - DNA, 24 bp, Artificial Sequence
  Description of Artificial Sequence Synthetic primer
ggctccatcttagccctagtcacg

SEQ ID NO: 3 - DNA, 26 bp, Artificial Sequence
  Description of Artificial Sequence Synthetic primer
agcactctctgcagtcatgcggctca

SEQ ID NO: 4 - DNA, 26 bp, Artificial Sequence
  Description of Artificial Sequence Synthetic primer
agcactctctgcagtctagcggctca

SEQ ID NO: 5 - DNA, 26 bp, Artificial Sequence
  Description of Artificial Sequence Synthetic primer
agcactctctgcagtcttgcggctca

SEQ ID NO: 6 - DNA, 26 bp, Artificial Sequence
  Description of Artificial Sequence Synthetic primer
agcactctctgcagtcaagcggctca

SEQ ID NO: 7 - DNA, 19 bp, Artificial Sequence
  Description of Artificial Sequence Synthetic probe
cggctagctgtgaaaggtc

SEQ ID NO: 8 - DNA, 21 bp, Artificial Sequence
  Description of Artificial Sequence Synthetic probe
cggctagctgtgaaaggtccg